(19) United States
(12) Patent Application Publication
(10) Pub. No.: US 2020/0209212 A1
(43) Pub. Date: Jul. 2, 2020

(54) MOBILE FACILITY FOR ANALYSING A FLUID

(71) Applicants: IFP Energies nouvelles, Rueil-Malmaison Cedex (FR); SEMM LOGGING, VESDUN (FR)

(72) Inventors: Bruno GARCIA, NEUILLY SUR SEINE (FR); Laurent BESSON, MONTLUCON (FR)

(21) Appl. No.: 16/621,639

(22) PCT Filed: May 15, 2018
(86) PCT No.: PCT/EP2018/062591

§ 371 Date: Dec. 11, 2019

(30) Foreign Application Priority Data
Jun. 21, 2017 (FR) 1755645

(51) Int. Cl. G01N 33/28; E21B 49/08

(52) U.S. Cl. CPC G01N 33/2823; E21B 2049/085; E21B 49/081

(57) ABSTRACT

The invention relates to a mobile fluid analysis system (1). System (1) is mobile so that it can be moved to the sampling site. To carry out the analysis, the system notably comprises at least two piston cells (3), PVT analysis means and geochemical analysis means (GEO). Furthermore, the invention relates to the use of such a mobile system (1) for analysing a fluid sample taken from an underground formation.

### FIELD OF THE INVENTION

The invention relates to the technical field of underground medium development, such as underground reservoir development (gas storage/withdrawal, hydrocarbon exploitation, geothermics, . . . ) and monitoring of these operations (contamination of operations on aquifers for example). The invention notably relates to the field of geological storage site monitoring for gases such as carbon dioxide (CO₂) or methane. The present invention also concerns other fields such as enhanced oil recovery for example or source rock gas exploitation.

In particular, the invention relates to a system for in-situ analysis of a fluid sample taken from an underground formation.

Fluids present in wells often need to be sampled in order to determine their composition so as to characterize the geological reservoirs reached by the well and their evolution over time during the industrial storage and/or production process. This is notably the case for geological gas storage site monitoring.

### BACKGROUND OF THE INVENTION

Industrialists have developed many techniques allowing the evolution of fluids injected into porous media to be monitored.

Geochemical monitoring methods for geological CO₂ storage sites, based on the study of volatile species, are for example known. Examples of these methods are described in patent applications FR-2,972,758 and FR-2,974,358.

These methods mainly apply for two compartments:

in reservoirs/saline aquifers where the main objective is to quantify the dissolved and precipitated CO₂, and thus to establish a real mass balance,

in aquifers overlying the cap rock, where the main objective is to diagnose a leak as early as possible.

To implement these methods, it is thus necessary to have a device for sampling fluids under pressure in a well drilled through a geological formation. Such a device is referred to as sampler.

Samplers referred to as FTS (Flow-Through Sampler), allowing to obtain fluid samples from a well drilled through a geological formation, are known. Such a device is comprised of a sample chamber with a spring-loaded valve at each end. A latching mechanism connects the valves together and holds them open. A clock for programming the closing time and a triggering mechanism for releasing the valves are arranged above the chamber. The lower end is provided with means allowing the fluid to enter. A rope socket for attaching a cable is arranged at the top.

French patent applications FR-299,224 and FR-3,011,029 also disclose sampling devices comprising, on the one hand, a piston controlled by a spring immersed in an oil chamber for sampling the fluid and, on the other hand, a second piston for expelling the fluid upon transfer. The device is maintained in open or closed position by the compressed spring housed in the oil-filled chamber. The oil contained in the spring chamber allows to dampen the decompression effect and to achieve smooth sampling. The device allows recovery of the fluid sample by means of the mechanical action of a solid piston through a manual valve. This device can also be advantageously lowered in open position into the underground medium, so as to avoid opening problems and to enable complete filling of the sampling chamber.

To implement these monitoring methods, it is then necessary to analyse the recovered fluid. Currently, the fluid collected with these samplers is analysed in an analysis laboratory that is often far away from the sampling site. This remoteness generates many drawbacks:
first, transport of the sample taken is likely to deteriorate the sample,
the need to take delivery of the sampler, which is considered to be dangerous equipment as it is under pressure,
customs-related or law issues when the analysis laboratory is in a different country than the sampling site,
a risk of contamination of the sampled fluid by a synthetic brine used to drive the fluid in place into the sampler, and
a long transport time, which prevents real-time analysis and therefore real-time underground site control, etc.

To overcome these drawbacks, the invention relates to a mobile fluid analysis system. The system is mobile so that it can be moved to the sampling site, thus avoiding the need to transporting the sampler for analysis. To perform the analysis, the system notably comprises at least two piston cells, PVT analysis means and geochemical analysis means. Thus, the system enables PVT analysis and geochemical analysis of the fluid to be analysed.

### SUMMARY OF THE INVENTION

The invention relates to a mobile fluid analysis system. Said mobile analysis system comprises at least:
a first circuit containing oil,
at least two piston cells, each piston cell including a piston delimiting two volumes, a first volume being connected to said first circuit,
connection means for connection to a container comprising said fluid to be analysed for transferring said fluid to be analysed into the second volumes of said piston cells,
a second circuit containing brine or water, notably pure water, said second circuit being connected to the container to control injection of said fluid to be analysed,
PVT analysis means for determining the gas-water ratio and/or the gas-oil ratio of said fluid to be analysed contained in a first piston cell,
geochemical analysis means for determining at least part of the composition of said fluid to be analysed contained in a second piston cell.

According to an embodiment of the invention, said geochemical analysis means comprise means for degassing said fluid contained in said second volume of said second piston cell.

According to an implementation, said first and second circuits comprise self-regulating pumps.

According to an aspect, said mobile analysis system comprises three piston cells, two of said piston cells being connected to geochemical analysis means.

Advantageously, said PVT analysis means comprise means for measuring the saturation pressure and the volume of the gases contained in said fluid to be analysed.

According to a feature, the mobile analysis system comprises a circuit for adding an inert gas, preferably nitrogen or argon, into said geochemical analysis means.

According to an embodiment, said geochemical analysis means comprise means for analysing the composition of the gases contained in said fluid to be analysed, said means being suited to determine the amount of said gases selected from among the following gases: CO₂, N₂, O₂, the hydrocarbons having C₁ to C₄ chains, He, Ne, Ar, Kr, Xe, and/or to determine the isotopic signature δ¹³C.

According to an implementation, said geochemical analysis means comprise a water trap and a sampling cylinder.

Advantageously, said mobile analysis system is contained in a substantially parallelepipedic casing of length less than or equal to 20 m, of height less than or equal to 3 m and of width less than or equal to 5 m.

Preferably, all the equipments of said mobile analysis system are stationary with respect to said casing.

According to an aspect, said mobile analysis system comprises a first set of valves for transferring said fluid to be analysed to said piston cells.

According to a feature, said mobile analysis system comprises a second set of valves for transferring said fluid to be analysed from said piston cells to said PVT analysis means and said geochemical analysis means.

According to an embodiment, said container of said fluid to be analysed is a downhole sampler.

The invention further relates to a use of a mobile analysis system according to one of the above features for analysing a fluid sample taken from an underground formation of a gas storage, hydrocarbon production, geothermal, natural hydrogen production site.

Advantageously, the saturation pressure, the volume of the gases present in the fluid to be sampled and the composition of the gases present in said fluid sample are analysed.

According to an embodiment, said mobile analysis system is moved to a sampling site where said fluid sample to be analysed is taken.

According to an implementation, the following steps are carried out:

a) installing a container with said fluid to be analysed in said mobile analysis system,

b) transferring said fluid to be analysed from said container to said piston cells,

c) carrying out a PVT analysis from said fluid contained in a first piston cell, and

d) carrying out at least one geochemical analysis from said fluid contained in at least a second piston cell.

### BRIEF DESCRIPTION OF THE FIGURES

Other features and advantages of the system according to the invention will be clear from reading the description hereafter of embodiments given by way of non limitative example, with reference to the accompanying FIGURE wherein:

FIG. 1 illustrates a fluid analysis system according to an embodiment of the invention.

### DETAILED DESCRIPTION OF THE INVENTION

The present invention relates to a mobile analysis system for a fluid, notably gases and dissolved gases in a fluid. The system is referred to as mobile because it can be moved from one site to another without dismantling the equipments. It is thus possible to move the system to a site where a fluid sample is taken in order to perform in-situ analyses of the fluid sample. The invention therefore affords notably the following advantages:
the mobile analysis system requires no transport or reception of the fluid sample,
the mobile analysis system allows to avoid customs-related or law issues,
the mobile analysis system limits risks of contamination of the fluid sample, and
the mobile analysis system allows quasi real-time analysis, which provides quasi real-time control of the underground site.

According to the invention, the mobile system comprises at least the following equipments:
a first circuit containing oil,
a second circuit containing brine or water, in particular pure water,
at least two piston cells, each cell including a piston delimiting two volumes: a first volume is connected to the first circuit containing oil and the second volume is connected to a container comprising the fluid to be analysed,
connection means for connection to the container comprising the fluid to be analysed for transferring the fluid to be analysed to one of the piston cells, in particular to the two volumes of the piston cells,
PVT (pressure, volume, temperature) analysis means for determining the gas-water ratio and/or the gas-oil ratio of the fluid to be analysed, the PVT analysis means are connected to a second volume of a first piston cell, and
geochemical analysis means for determining at least partly the composition of said fluid to be analysed contained in a second volume of at least one of the piston cells.

Thus, to carry out the analyses of the fluid to be analysed, the fluid is transferred from its container to the piston cells, the piston cells being first empty, then from the piston cells to (PVT and geochemical) analysis means. The piston cells allow to homogenize the overhead volume obtained in case of a small amount of gas present in the fluid to be analysed (by adjusting the pressure in the piston cells). Furthermore, the piston cells allow to adjust the injection pressures for the (PVT and geochemical) analyses, by adjusting the pressures of the fluids in the first and second circuits.

The invention allows to carry out various in-situ analyses of the fluid to be analysed.

The second circuit is connected to the container so as to control injection of the fluid to be analysed in the measurement cells. Optionally, the second circuit can be connected to the connection means to allow purge of the system.

According to an aspect of the invention, the piston cells can have a volume ranging between one sixth and one third of the volume of the container comprising the fluid to be analysed. According to a non-limitative example, the volume of the container comprising the fluid to be analysed can be substantially 600 cm³, and the system can include three piston cells with a respective volume of substantially 150 cm³.

Preferably, the fluid to be analysed can be a fluid sample taken from an underground formation, in particular a gas storage site, a hydrocarbon production site (conventional or unconventional, using an enhanced oil recovery EOR method or not), a geothermal site, a natural hydrogen production site, or any similar site. The fluid sample taken from an underground formation and intended to be analysed may come in form of a brine, i.e. an aqueous solution with a high salt concentration. Furthermore, the fluid sample can comprise hydrocarbons. Alternatively, the fluid to be analysed can be of any other nature.

Advantageously, the container comprising the fluid to be analysed can be a downhole sampler. It is thus possible to directly connect the fluid sample to be analysed with the analysis system without container transportation and without fluid transfer from the downhole sampler to an intermediate container. The downhole sampler can notably correspond to one of the downhole samplers described in patent applications FR-2,999,224 and FR-3,011,029. Alternatively, the container may be of any other nature.

Advantageously, to perform the geochemical measurements, the geochemical analysis means can include means for degassing the fluid contained in the second volume of a piston cell. These degassing means can consist of means for controlling the pressure in the piston cell. Indeed, by decreasing the pressure within the piston cell, the fluid to be studied is degassed.

Furthermore, in order to facilitate measurement, if the volume of gas present in the fluid is low, the facility can comprise a circuit for adding an inert gas, upstream from the geochemical analysis means. The inert gas added can be an inert gas or a rare gas such as nitrogen or argon.

According to an implementation of the invention, the geochemical analysis means can comprise means for analysing the composition of the gases present in the fluid to be analysed, these means being suited to determine the amount of said gases selected from among the following gases: CO₂, N₂, O₂, the gaseous hydrocarbons having C₁ to C₄ chains, He, Ne, Ar, Kr, Xe, and/or to determine the isotopic signature δ¹³C. It is thus possible to determine the composition of the gases generally present in the fluid to be analysed, in particular when the fluid sample has been taken from an underground formation.

According to an embodiment of the invention, the geochemical analysis means can include a water trap allowing to isolate the gas to be analysed. Furthermore, the geochemical analysis means can comprise a sampling cylinder for performing the corresponding quantitative measurements. The measurements can be performed with a conventional analyser that may be connected and integrated in the system.

Advantageously, the PVT analysis means can comprise means for measuring the saturation pressure of the gases present in the fluid to be analysed and the volume of the gases present in the fluid to be analysed. The gas-water ratio and/or the gas-oil ratio of the fluid to be analysed can be determined by means of the volume measurements. The saturation pressure allows to know the bubble point of the gases present in the fluid to be analysed.

According to an embodiment of the invention, the PVT analysis is carried out prior to the geochemical analyses. The volume of fluid required for the geochemical analyses can thus be adjusted, qualitatively as well as quantitatively.

In order to automate the analyses, the first and second circuits may include self-regulating pumps.

Furthermore, in order to control fluid transfers between the container and the piston cells, the system can comprise a first set of valves. These valves are controlled so as to carry out the steps allowing the various measurements to be performed.

Besides, to control fluid transfers between the piston cells, the PVT analysis means and the geochemical analysis means, the system can include a second set of valves. These valves are controlled so as to carry out the steps allowing the various measurements to be performed.

According to an implementation of the invention, the system can comprise three piston cells. For this implementation, one piston cell can be connected to PVT analysis means, and two piston cells can be connected to distinct geochemical analysis means. This design allows sampling to be doubled.

In a variant, the mobile analysis system can comprise between 2 and 6 piston cells.

In order to be easily transported, the mobile system can be contained in a substantially parallelepipedic casing. For example, the mobile analysis system can be contained in a site cabin or in a container used notably for sea freight. The casing can have a length less than or equal to 8 m, a height less than or equal to 2 m and a width less than or equal to 3 m.

For this implementation, the equipments of the mobile analysis system can be stationary with respect to the casing.

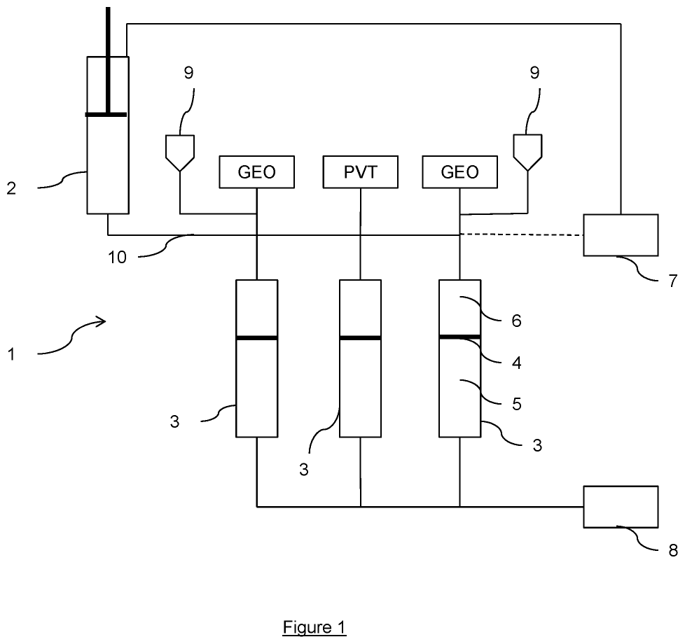

FIG. 1 schematically illustrates, by way of non-limitative example, a mobile analysis system according to an embodiment of the invention. In this FIGURE, the control means and the valves enabling transfer are not shown. Mobile analysis system 1 comprises a container 2 containing the fluid to be analysed. Container 2 comprises fluid transfer means: a piston, according to the example illustrated. Container 2 can be a downhole sampler. The mobile analysis system 1 illustrated comprises three piston cells 3. Each piston cell 3 comprises a piston delimiting two volumes 5 and 6. First volume 5 is connected to a first circuit 8 containing oil. Second volume 6 is connected to container 2 by connection means 10. Furthermore, the system comprises a second circuit 7 containing brine or water, pure water for example. Second circuit 7 is connected to container 2 so as to control injection of the fluid to be analysed into the measurement cells. Optionally, second circuit 7 can be connected to connection means 10 to allow purge of the system. The first 8 and second 7 circuits include self-regulating pumps (not shown). Central piston cell 3 is connected to PVT analysis means for determining the gas-water ratio and/or the gas-oil ratio of the fluid to be analysed. The other two piston cells 3 are connected to geochemical analysis means GEO for analysing at least partly the composition of the fluid to be analysed. According to the embodiment illustrated, mobile system 1 optionally comprises means for adding an inert gas 9 upstream from the geochemical analysis means. For example, the inert gas may be nitrogen or argon.

Furthermore, the invention relates to the use of a mobile analysis system according to any one of the variant combinations described above for analysing a fluid sample taken from an underground formation of a gas storage site (CO₂ for example), a hydrocarbon production site (conventional or unconventional, using an enhanced oil recovery EOR method or not), a geothermal site, a natural hydrogen production site, or any similar site. It may notably be the mobile analysis system schematically illustrated in FIG. 1.

Use of the mobile system can be implemented within the context of the monitoring of one of these sites, and it may notably allow to detect fluid leakage in an underground formation. After detecting a leakage, remedial action can be rapidly taken to stop the leakage, by means of the in-situ analyses. For example, in case of gas storage in the underground formation, a stored gas leakage may be detected, and it is then possible to limit the amount of stored gas to prevent larger leakage.

The mobile system enables to analyse the saturation pressure, as well as the gas-water and gas-oil ratios of the gases present in the fluid sample (by means of the PVT analysis means). The composition of the fluid sample can also be analysed (by means of the geochemical analysis means).

The system being mobile, the use of this mobile analysis system can comprise a step wherein the mobile analysis system is directly moved to (close to) the fluid sampling site.

According to an embodiment of the invention, the mobile analysis system can be used by carrying out the following steps:

a) installing a container with the fluid to be analysed in said mobile analysis system,

b) transferring the fluid to be analysed from the container to the piston cells,

c) carrying out a PVT analysis from the fluid contained in a first piston cell so as to determine the gas-water ratio and/or the gas-oil ratio of the fluid to be analysed, and

d) carrying out at least one geochemical analysis from the fluid contained in at least a second piston cell so as to determine at least partly the composition of the fluid to be analysed.

Using the mobile analysis system can comprise a prior step of taking a sample of a fluid to be analysed from an underground formation.

In step a), the container can be a downhole sampler used for taking a fluid sample from an underground formation.

In step c), the saturation pressure and the volume of the gases present in the fluid to be analysed are measured by means of the PVT analysis.

According to an embodiment of the invention, the PVT analysis is carried out prior to the geochemical analyses, and the volume of fluid required for the geochemical analyses is adjusted, qualitatively as well as quantitatively.

In step d), the geochemical analysis allows to determine the amount of said gases selected from among the following gases: CO₂, N₂, O₂, the gaseous hydrocarbons having C₁ to C₄ chains, He, Ne, Ar, Kr, Xe, and/or the isotopic signature δ¹³C. It is thus possible to determine the composition of the gases generally present in the fluid to be analysed, in particular when it is a fluid sample taken from an underground formation.

According to an embodiment of the invention, the use can comprise a step of adding an inert gas, such as nitrogen or argon, to the fluid to be analysed prior to geochemical analysis step d). This addition allows to facilitate measurement when the volume of gas to be analysed is low.

The use according to the invention can also comprise a step of preparing the system. In this step, the piston cells can be purged so as to remove the fluids that may be initially present, by moving the piston of the piston cells notably by means of the oil of the first circuit. The piston cells can then be saturated with brine by means of the second circuit. Finally, the pressure within the piston cells is regulated by adjusting the pressure of the first and second circuits.

1. A mobile fluid analysis system, wherein the mobile analysis system comprises at least:
a first circuit containing oil,
at least two piston cells, each piston cell including a piston delimiting two volumes, a first volume being connected to the first circuit,
connection means for connection to a container comprising the fluid to be analysed for transferring the fluid to be analysed into the second volumes of the piston cells,
a second circuit containing brine or water, notably pure water, the second circuit being connected to container to control injection of the fluid to be analysed,
PVT analysis means (PVT) for determining the gas-water ratio and/or the gas-oil ratio of the fluid to be analysed contained in a first piston cell,
geochemical analysis means (GEO) for determining at least part of the composition of the fluid to be analysed contained in a second piston cell.

2. A system as claimed in claim 1, wherein the geochemical analysis means (GEO) comprise means for degassing the fluid contained in the second volume of the second piston cell.

3. A system as claimed in claim 1, wherein the first and second circuit comprise self-regulating pumps.

4. A system as claimed in claim 1, wherein the mobile analysis system comprises three piston cells, two of the piston cells being connected to geochemical analysis means (GEO).

5. A system as claimed in claim 1, wherein the PVT analysis means comprise means for measuring the saturation pressure and the volume of the gases contained in the fluid to be analysed.

6. A system as claimed in claim 1, wherein mobile analysis system comprises a circuit for adding an inert gas, preferably nitrogen or argon, into the geochemical analysis means (GEO).

7. A system as claimed in claim 1, wherein the geochemical analysis means (GEO) comprise means for analysing the composition of the gases contained in the fluid to be analysed, the means being suited to determine the amount of the gases selected from among the following gases: CO₂, N₂, O₂, the hydrocarbons having C₁ to C₄ chains, He, Ne, Ar, Kr, Xe, and/or to determine the isotopic signature δ¹³C.

8. A system as claimed in claim 1, wherein the geochemical analysis means (GEO) comprise a water trap and a sampling cylinder.

9. A system as claimed in claim 1, wherein the mobile analysis system is contained in a substantially parallelepipedic casing of length less than or equal to 20 m, of height less than or equal to 3 m and of width less than or equal to 5 m.

10. A system as claimed in claim 9, wherein all the equipments of the mobile analysis system are stationary with respect to the casing.

11. A system as claimed in claim 1, wherein the mobile analysis system comprises a first set of valves for transferring the fluid to be analysed to the piston cells.

12. A system as claimed in claim 1, wherein the mobile analysis system comprises a second set of valves for transferring the fluid to be analysed from the piston cells to the PVT analysis means and the geochemical analysis means (GEO).

13. A system as claimed in claim 1, wherein the container of the fluid to be analysed is a downhole sampler.

14. Use of a mobile analysis system as claimed in claim 1, for analysing a fluid sample taken from an underground formation of a gas storage, hydrocarbon production, geothermal, natural hydrogen production site.

15. Use as claimed in claim 14, wherein the saturation pressure, the volume of the gases present in the fluid to be sampled and the composition of the gases present in the fluid sample are analysed.

16. Use as claimed in claim 14, wherein the mobile analysis system is moved to a sampling site where the fluid sample to be analysed is taken.

17. Use as claimed in claim 14, wherein the following steps are carried out:
a) installing a container with the fluid to be analysed in the mobile analysis system,
b) transferring the fluid to be analysed from the container to the piston cells,
c) carrying out a PVT analysis from the fluid contained in a first piston cell, and
d) carrying out at least one geochemical analysis (GEO) from the fluid contained in at least a second piston cell.
